(19) United States
(12) Patent Application Publication
(10) Pub. No.: US 2004/0254624 A1
(43) Pub. Date: Dec. 16, 2004

(54) Electrical stimulator and garment electrode connection system

(75) Inventor: James P. Johnson, Gainesville, GA (US)

Correspondence Address: GARDNER GROFF, P.C., PAPER MILL VILLAGE, BUILDING 23, 600 VILLAGE TRACE, SUITE 300, MARIETTA, GA, 30067, US

(73) Assignee: Prizm Medical, Inc., Duluth, GA

(21) Appl. No.: 10/862,120

(22) Filed: Jun. 4, 2004

Related U.S. Application Data
(60) Provisional application No. 60/476,351, filed on Jun. 6, 2003.

(51) Int. Cl. A61N 1/18

(52) U.S. Cl. 607/149; 607/48

(57) ABSTRACT

An electronic stimulator for delivery of energy to a treated body portion through a garment or other form of electrode, and a method of treatment using said stimulator. A connector strap comprising diode bridge circuitry maintains proper polarity on the electrodes regardless of the orientation of connection between the stimulator and the electrode.

### CROSS-REFERENCE TO RELATED APPLICATIONS

This application claims priority to U.S. Provisional Patent Application Ser. No. 60/476,351, filed Jun. 6, 2003, which is hereby incorporated by reference herein for all purposes. U.S. Pat. Nos. 4,554,923; 4,664,118 and 5,374,283; and U.S. patent application Ser. No. 10/460,084 filed Jun. 12, 2003 (Publication No. US-2004-0030270-A1), are also hereby incorporated by reference herein for all purposes.

### FIELD OF THE INVENTION

The present invention relates generally to an electrical stimulator, and to connectors and therapeutic garment systems used in connection therewith. More particularly, the invention relates to a pulsed electrical stimulator for direct attachment onto a garment electrode, for providing electrical stimulation to nerve, muscle, skin, circulatory and/or other tissue of a human or animal subject.

### BACKGROUND OF THE INVENTION

Electrical stimulation has been found to provide therapeutic benefit to various biological tissue. For example, transcutaneous electrical nerve stimulation (TENS) has been used to treat neuromuscular injuries and other conditions by stimulation of muscle and nerve tissue. Electrical stimulation has also been found to increase circulation, promoting faster healing of injuries and preventing adverse conditions resulting from poor circulation. For example, many diabetics suffer from poor circulation in their extremities. Foot ulceration caused by poor circulation often progresses to more severe tissue damage and even loss of limb in many subjects. These conditions are commonly aggravated as a result of venous stasis or impaired function of venous valves in the extremities.

Electrical stimulation may be delivered to treated tissue by one or more electrodes, such as adhesive patch electrodes or garment electrodes. U.S. Pat. Nos. 4,554,923 ; 4,664,118 and 5,374,283 disclose various forms of garment electrodes for applying electrical stimulation from an electronic stimulator to a treated body part. U.S. patent application Ser. No. 10/460,084 filed Jun. 12, 2003 (Publication No. US-2004-0030270-A1) discloses a garment electrode capable of applying compression, preferably gradient compression, to a treated body part. Compression applied in combination with electrical stimulation has been found to provide many benefits for human and/or animal patients with venous disease or related and similar conditions.

Many previously known electrical stimulators have been found lacking in one or more aspects. For example, many known stimulators are large, heavy and unwieldy to connect to the stimulator electrodes used in connection therewith, and tend to be uncomfortable in use. And some known stimulators have been found to be unduly complex in their construction and use, resulting in undue expense and the potential for incorrect application, as for example by misconnection of electrical contacts. Also, known electrical stimulators are typically worn attached to the user's belt, and long wire leads are connected to transmit the electrical energy signal to electrodes attached to remote locations on the user's body. These wire leads can be inconvenient to connect, and can limit the user's motion or become disconnected as the user moves. Also, known means of connection between the stimulator and the electrodes must be carefully connected to provide proper polarity of the delivered stimulation energy, and therapeutic benefit may be impaired if the polarity is reversed due to inadvertent misconnection of the leads.

Thus it can be seen that needs exist for improvements in electrical stimulation devices and therapies, and to improved garment electrode connection systems used in connection therewith. It is to the provision of devices, systems and methods meeting these and other needs that the present invention is primarily directed.

### SUMMARY OF THE INVENTION

In its various embodiments, the present invention provides an improved stimulator and garment electrode system for delivering therapeutic energy to treated tissue of a human or animal subject. For example, in one aspect, the invention is an electrical stimulator for use in connection with garment electrodes and other electrodes for affixation to the body of a human or animal subject. The stimulator of the present invention preferably comprises an energy source and means for applying energy from the energy source to the treated body portion. The electrical stimulator is preferably relatively small and lightweight, and is simple in construction and use thereby reducing cost and preventing inadvertent misuse as by improper connection of electrical contacts.

In example embodiments, the electrical stimulator of the present invention is particularly adapted for direct attachment onto a garment electrode worn by the wearer, thereby eliminating the need for long wire leads extending from a belt-worn stimulator to a remote electrode position.

In further example embodiments, the stimulator of the present invention is programmable, enabling use of various treatment regimens. In still further embodiments, the stimulator includes sensors or other input means and onboard memory, for collecting and storing biometric data and patient compliance information; and/or a transmitter, display or other output means for monitoring by the subject and/or a medical caregiver.

In another aspect, the present invention is an improved therapeutic system for delivering energy to a treated body portion of a human or animal subject, the system including an electrode for application to the treated body portion, and an electronic stimulator attached directly to the electrode, the electronic stimulator delivering energy to the electrode. The electrode may for example be a garment electrode such as a stocking, sleeve, wrap, glove, or other type of garment to be worn over one or more body parts of a human or animal subject, at least a portion of the garment comprising a conductive material for delivering therapeutic energy to a treated body part. Optionally, the garment electrode is a compression garment, which further optionally provides gradient compression.

In another aspect, the invention is a therapeutic stimulator including an energy source, at least one electrical connection for delivering energy from the energy source to an electrode, and means for direct attachment of the stimulator to the electrode.

In still another aspect, the invention is a connector for delivering energy from an electronic stimulator to a treated body portion of a human or animal subject, comprising a flexible strap, and a diode bridge within said strap for maintaining proper polarity of the delivered energy.

In another aspect, the invention is a connector strap for providing electrical connection between an electronic stimulator and a pair of electrodes. The connector strap preferably includes first and second inner contacts, and first and second outer contacts. The connector strap preferably also includes a first conductor path between the first inner contact and the first outer contact, the first conductor path including a first diode; a second conductor path between the first inner contact and the second outer contact, the second conductor path including a second diode; a third conductor path between the second inner contact and the first outer contact, the third conductor path including a third diode; and a fourth conductor path between the second inner contact and the second outer contact, the fourth conductor path including a fourth diode.

In yet another aspect, the invention is a method of treating a body portion of a human or animal subject, the method including steps of applying a garment electrode to a body portion to be treated, and delivering therapeutic energy to the electrode from an electronic stimulator attached directly to the electrode. Optionally, the stimulator is connected to the garment electrode using connecting means for maintaining proper polarity of energy delivered therethrough. The garment electrode optionally also provides compression, and more preferably gradient compression.

In particularly preferred embodiments, the garment electrode and connection system of the present invention are used in combination with the electrical stimulator of the present invention, to form a therapeutic system for improving circulation and/or enhancing healing. It will be recognized, however, that the garment electrode connection system and the electrical stimulator of the present invention have separate and individual utility, independent of one another, as well as in combination.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 shows a perspective view of an electrical stimulator and therapeutic garment connection system according to an example embodiment of the present invention.

FIG. 2 shows a front elevational view of an electrical stimulator according to an example embodiment of the present invention.

FIG. 3 shows a rear elevational view of the electrical stimulator of FIG. 2.

FIG. 4 shows a side view of the electrical stimulator of FIG. 2.

FIGS. 5a-5d show a connector strap component according to an example embodiment of the present invention.

FIG. 6 shows a therapeutic stimulation waveform delivered according to an example form of the present invention.

### DETAILED DESCRIPTION OF PREFERRED EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

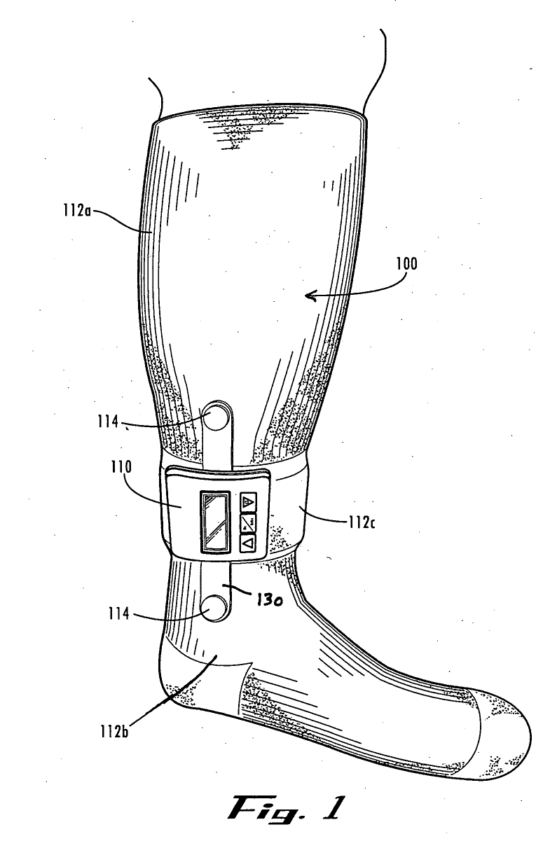

FIG. 1 shows a system according to an example form of the present invention, including a garment electrode 100 having an electrical stimulator 110 directly attached thereto by a connector strap. The garment electrode is depicted as a stocking, but other embodiments of the invention include a sleeve, wrap, glove, or other type of garment electrode to be worn over one or more body parts of a human or animal subject. The garment 100 preferably comprises one or more electrically conductive portions. For example, fibers, loops, rings, strips, panels, links or other elements of silver, copper, gold, other metals, and/or other conductive materials can be incorporated into the garment. Alternatively, non-conductive fibers or other elements of the garment 100 can be impregnated with a conductive material, coated with a conductive material, or otherwise rendered conductive. The garment 100 or one or more portion(s) thereof is alternatively formed of non-conductive knitted microfiber fabric or other non-conductive material and is plated on one or more faces with a conductive material, or otherwise rendered electrically conductive. In preferred embodiments, the garment electrode 100 preferably comprises a first conductive region 112a to apply therapeutic energy having a first electrical polarity, and a second conductive region 112b to apply energy with a second electrical polarity opposite the first polarity; with the first and second conductive regions 112a, 112b separated by a non-conductive region 112c.

One or more metallic snap connectors, permanent or detachable electrical couplings, and/or other type of connection means is/are preferably provided on the electrode 100 for attaching the electrical stimulator 110 to the electrode. The stimulator 110 and/or an intermediate connector strap component preferably comprise(s) two or more cooperating contacts 114, such as for example metallic snap connectors, for engaging and forming an electrically conductive connection with cooperating connection means of the electrode 112. In the embodiment shown in FIG. 1, an intermediate connector strap 130 is permanently or detachably connected to the stimulator, and is detachably connected to the garment electrode by snap connectors 114.

The stimulator and connection system of the present invention are also suitable for use in combination with adhesive single-pole patch electrodes and/or other varieties of electrodes. However, it has been discovered that even localized tissue wounds, such as for example diabetic foot ulcers, are generally symptomatic of circulatory problems in a wider overall area of the body, and that treatment of the entire region of poor circulation is therapeutically more effective at healing the wound and preventing further damage than localized treatment of the wound area only. Without being in any way limited or bound by theory, it is believed that the use of a garment electrode generates an electromagnetic field in and around substantially an entire body portion upon which the garment is worn, stimulating improved circulation throughout substantially that entire body region and promoting healing and prevention of new injury. The Silver-Thera™ (MicroZ™) garment electrodes available from Prizm Medical Inc. of Duluth Ga. are suitable for use in this regard.

The garment 100 is preferably at least partially elastic, and is sized and shaped to provide compression when worn on the intended body part(s) of the subject. For example, garments according to the present invention can be provided in a range of sizes and garment configurations for subjects of virtually any anticipated size and shape. In further preferred and optional embodiments, at least a portion of the garment 100 is sized, shaped and configured to provide gradient compression when worn by the subject. For example, a stocking can be knit to provide a greater degree of compression toward the subject's toes, and progressively lesser degrees of compression toward the upper open end of the stocking. Gradient compression can be achieved by varying the fiber content or knit of the garment, and/or by tapering the shape of the garment to provide a tighter fit (i.e., greater compression) in one portion and a looser fit (i.e., less compression) in another portion. In other embodiments of the invention, a conductive garment without compression features may be applied to a body part in combination with a standard non-conductive compression or gradient compression garment, for example by layering the compression garment over the conductive garment. For ease of use, the compression garment is optionally permanently or releasably attached to the conductive garment in their layered configuration, as by stitching, adhesive, snaps, buttons, hook-and-loop materials, and/or other attachment means.

Compression in medical garments helps promote stability, increases blood flow and reduces edema. Various injuries or diseases to the lower extremities, as well as to the ankle, knee, hand/wrist, elbow, etc., can benefit from compression in medical garments. For example, gradient compression socks and stockings are used to treat poor circulation in the leg. The function of gradient compression hosiery is to deliver pressure at the ankle that gradually decreases as it extends up the leg, helping to move blood back to the heart. This activity reduces blood pooling in the legs. The application of electrotherapy in combination with compression is believed to provide improved degrees of blood flow promotion, edema reduction, inflammation reduction and pain reduction as compared to electrical stimulation or compression applied independent of one another. For example, during the resting phase of circulation under compression, electrical stimulation is believed to assist in the necessary inflow of blood into the deep venous system.

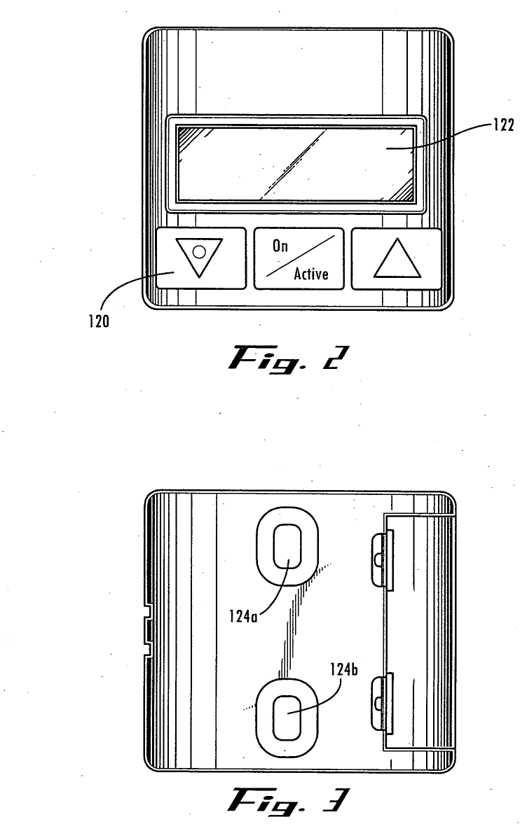

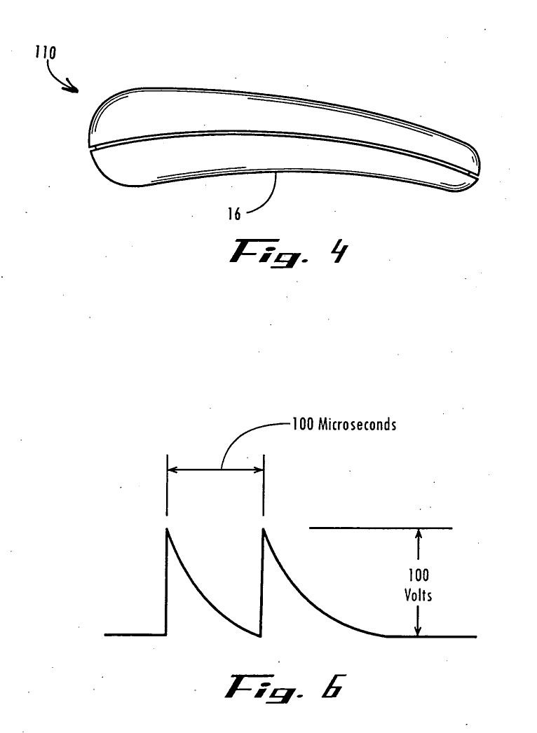

FIGS. 2-4 show more detailed views of the stimulator 110. The stimulator 110 preferably comprises a housing having a concavely curved inside (or rear) face 116 for conforming to an arm, leg or other curved body portion of a wearer, for improved comfort and stability when directly attached to a garment electrode worn on the body part. For example, the concavely curved face preferably has a radius of curvature of between about 2″ to about 6 ″, and more preferably of about 4″. The stimulator 110 is preferably thin, small and lightweight, relative to most previously known electronic stimulators. For example, the height and width of the stimulator 110 are preferably less than about 3″, and more preferably no more than about 2″ high by about 2.5″ wide; the thickness is preferably less than about 0.75″, and more preferably no more than about 0.65″; and the weight of the stimulator is less than about 3.0 ounces, and more preferably no more than about 2.0 ounces.

The stimulator 110 preferably includes one or more control pads or buttons 120 for actuating the device and inputting commands, and one or more displays or outputs 122. The stimulator 110 preferably includes an energy source, such as a battery, and a processor, memory and control circuitry, and resident software for receiving and processing input data, processing and controlling the output signal of the stimulator, delivering the output signal to the electrodes, and/or displaying output data. In an example embodiment, two standard AAA batteries comprise the energy source. The stimulator 110 preferably further includes one or more externally accessible coupling portions 124, such as terminals or contacts, for permanent or detachable electrically conductive connection to cooperating connector portions of the electrode(s), and/or of a connector strap or leads extending between the stimulator and the electrode(s). For example, electrically conductive snap connectors 124 are depicted in the drawing figures. The snaps or other cooperating connector portions preferably provide a secure means of direct attachment of the stimulator 110 onto the garment electrode 110. As used herein, “direct” attachment of the stimulator to the electrode refers to the physical carrying or mounting of the stimulator onto or into the electrode, either by direct engagement of the stimulator to the electrode or engagement via an intermediate connector strap or other connection member, regardless of whether the electronic connection between the stimulator and the electrode is direct, via one or more intermediate conductors, via wireless signal, or otherwise. Alternatively or additionally, one or more other forms of attachment means can be provided for direct attachment of the stimulator to the garment electrode, such as for example hook and loop fasteners, clips, snaps, hook and eyelet connectors, straps, pockets, etc.

The stimulator 110 preferably delivers electrical stimulation from an energy source such as a battery to the garment or other type of electrode according to a prescribed stimulation sequence or regimen. Internal circuitry within the stimulator controls the delivery of energy to the subject and controls the parameters (e.g., current, voltage, waveform, frequency, duration, cycle parameters, etc.) of the delivered energy. In example embodiments, the control circuitry is substantially similar or identical to that of known commercially-available electronic stimulators, or can be modified for the specific purposes described herein, as is within the capabilities of those of ordinary skill in the art. For example, microamperage stimulation can be delivered in a high-volt pulsed galvanic monophasic exponential spike wave form, and/or twin-peak monophasic wave form, with 100V peak voltage and 100 microsecond peak-to-peak pulses, as shown in FIG. 6. In alternate treatment regimens, the peak voltage delivered is greater or less than 100V, for example between about 24V to about 200V; and the peak-to-peak interval is greater or less than 100 microseconds, for example between about 25 microseconds to about 400 microseconds. In example embodiments, the stimulator is controllable to provide treatment times of between about 15 minutes to about 24 hours, to deliver pulse rates of between about 1 pulse per second to about 120 pulses per second in 5V (or other) increments from between about 0V to about 200V, and for positive or negative polarity energy delivery. While high-voltage, low-amperage pulsed galvanic stimulation is described in detail above and depicted in the figures, alternate embodiments of the invention encompass the delivery of other energy forms such as noise signals (e.g., sound waves), vibration, light or other electromagnetic energy, physical manipulation, magnetism, etc., from the stimulator to a garment or other type of electrode or transducer, instead of or in addition to the delivery of electrical stimulation, to improve circulation, enhance healing and/or prevent injury. For example, alternating sequences of noise and electrical stimulation can be delivered to a target body portion of a human or animal subject according to alternate forms of the invention.

In further embodiments, the stimulator 110 optionally comprises memory, programming and/or reporting capacity. For example, a serial port or infrared wireless transmitter can be provided for communication between the stimulator 110 and a docking station, computer workstation, printer, display, programmer or other input/output means. The user or a medical caregiver inputs a prescribed treatment regimen or selects from a pre-programmed list of treatment regimen options. In an example embodiment, the stimulator 110 is preprogrammed with at least two treatment regimen options that can be selected, such as:

Program 1—a 30-minute cycle with 15 minutes of stimulation at 100 Hz followed by 15 minutes at 10 Hz;

Program 2—an 8-hour cycle with 10 minutes of stimulation at 80 Hz, followed by 10 minutes at 8 Hz, followed by 40 minutes without stimulation, repeated eight times.

In further embodiments, the cycle parameters are separately controlled by inputting the desired frequency, time, voltage, etc. The stimulator 110 optionally includes onboard memory for storing data such as compliance and diagnostic information that can be accessed and/or downloaded for analysis by the user or a medical caregiver to monitor the use and compliance of the device and assist in diagnosis and monitoring of treatment progress. For example, the stimulator may include one or more transducers for measuring the skin temperature of the subject to monitor circulation, electrical resistance, current flow, and other variables of potential relevance. The stimulator and/or a separate docking port or other peripheral device optionally also include(s) communication means such as a wireless communication system or modem for communicating input and output data over a communications network such as the Internet or a telephone line, so that a medical caregiver can exchange data, program input and/or monitor output to/from the stimulator from a remote location. Output intended for use by a medical caregiver is optionally delivered in the form of an alphanumeric code, or otherwise encoded in a manner that would prevent the user from understanding and/or tampering with such data.

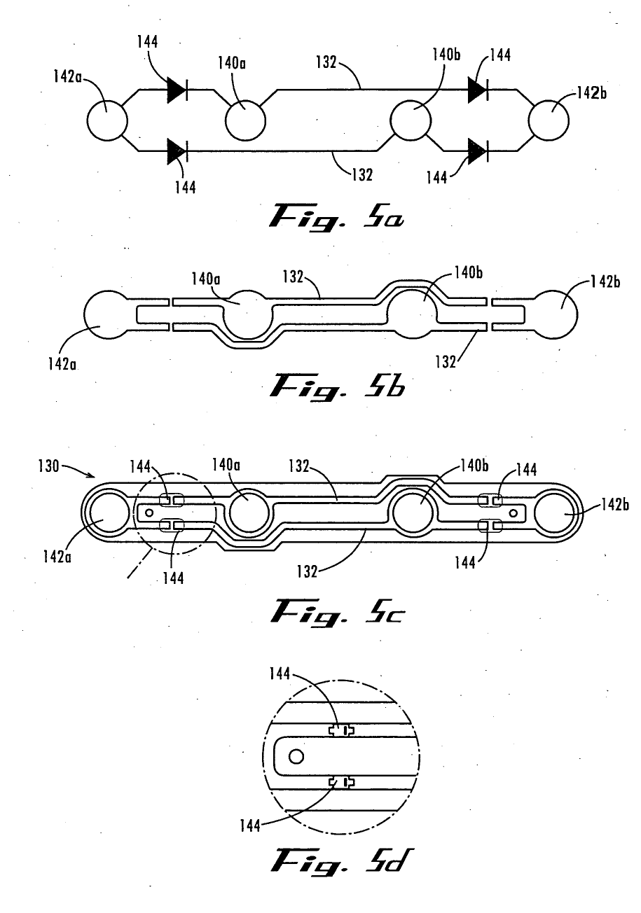

With reference to FIGS. 1 and 5a-5d, the present invention preferably also includes a connector strap 130 for providing an electrically-conductive connection between the electrical contact(s) or terminal(s) of an electrical stimulator (such as, but not limited to the described stimulator 110) and the snap connector(s) or other electrical contact(s) of a conductive garment or other form of electrode. The strap 130 takes the place of the connector cords or wire leads typically used for making an electrical connection between a stimulator and electrode(s). The strap can be permanently attached to the stimulator, or can be a separate component suitable for connection to various stimulator types. The strap 130 is preferably a flexible element having one or more conductors 132 covered by or embedded within a covering 134 of vinyl or other flexible non-conductive material(s), and preferably comprises a thin and generally flat belt or strip of material. In further embodiments of the invention, the strap 130 comprises “smart” circuitry, such as a diode bridge (see circuit diagram of FIG. 5a) for maintaining proper polarity regardless of the orientation of the stimulator. Inner contacts 140a, 140b of the connector strap are connected (as by metallic snaps and/or other electrically conductive connectors) to corresponding terminals or contacts on the stimulator without regard to their polarities, and outer contacts 142a, 142b of the connector strap are connected (also as by metallic snaps and/or other electrically conductive connectors) to corresponding terminals on the garment or electrode. Because of the configuration of diodes 144, which are either “on” to permit intended current flow or off to prevent unintended counter-flow or current, electrical current can only flow in one direction on the conductor paths 132, thereby maintaining the proper polarity of charge on the garment or other electrodes regardless of the orientation and polarity of the stimulator.

The present invention further comprises a treatment method for delivering energy to a body portion of a human or animal subject. In example embodiments, the treatment is carried out during sleep, when most healing naturally occurs. Without being in any way limited or bound by theory, it is believed that growth hormone activity in the body is naturally more active during resting periods, and that healing therefore takes place more rapidly during these periods. However, bloodflow to the extremities is believed to slow during these periods to maintain core body temperature during inactivity. Delivery of electrical stimulation to one or more portions of the body, and in particular the extremities, is believed to increase bloodflow in these body portions to enhance the healing process. In example embodiments, the treatment method of the present invention includes debridement of dead or affected tissue, lessening or removing load on the treated area (for example, by using a cane, crutch, walker or wheelchair to offload an affected leg), wound care by cleaning and/or use of antibiotics, and electrical stimulation or otherwise applying energy to enhance circulation in the treated area and/or surrounding tissue. In example forms, the stimulation is applied in cycles, for example by providing stimulation for about twenty minutes in each hour.

While the invention has been described with reference to preferred and example embodiments, it will be understood by those skilled in the art that a number of modifications, additions and deletions are within the scope of the invention, as defined by the following claims.

What is claimed is:

1. A system for delivering energy to a treated body portion of a human or animal subject, comprising:
an electrode for application to the treated body portion; and
an electronic stimulator attached directly to the electrode, said electronic stimulator delivering energy to the electrode.

2. The system of claim 1, wherein the electrode is a garment electrode.

3. The system of claim 2, wherein the garment electrode comprises a glove.

4. The system of claim 2, wherein the garment electrode comprises a sock.

5. The system of claim 2, wherein the garment electrode comprises a sleeve.

6. The system of claim 2, wherein the garment electrode comprises at least two conductive portions, said conductive portions being separated from one another by a non-conductive portion.

7. The system of claim 2, wherein the garment electrode provides compression to the treated body portion.

8. The system of claim 7, wherein the garment electrode provides gradient compression to the treated body portion.

9. The system of claim 1, wherein the electronic stimulator comprises a curved inner face for placement against the electrode.

10. The system of claim 1, wherein the electronic stimulator has a height and a width of no more than about 2.5 inches.

11. The system of claim 1, wherein the electronic stimulator has a thickness of no more than about 0.65 inches.

12. The system of claim 1, wherein the electronic stimulator has a weight of no more than about 2.0 ounces.

13. The system of claim 1, wherein the electronic stimulator is attached to the electrode by a connector strap.

14. The system of claim 13, wherein the connector strap comprises a diode bridge for maintaining an intended polarity of the energy delivered to the electrode.

15. A therapeutic stimulator comprising an energy source, at least one electrical connection for delivering energy from said energy source to an electrode, and means for direct attachment of said stimulator to the electrode.

16. The therapeutic stimulator of claim 15, further comprising an outer housing having a curved inner face for placement against the electrode.

17. The therapeutic stimulator of claim 15, having a height and a width of no more than about 2.5 inches.

18. The therapeutic stimulator of claim 15, having a thickness of no more than about 0.65 inches.

19. The therapeutic stimulator of claim 15, having a weight of no more than about 2.0 ounces.

20. The therapeutic stimulator of claim 15, wherein said means for direct attachment of said stimulator to the electrode comprises a connector strap.

21. The therapeutic stimulator of claim 20, wherein the connector strap also provides the at least one electrical connection for delivering energy from said energy source to an electrode.

22. The therapeutic stimulator of claim 21, wherein the connector strap comprises a diode bridge for maintaining an intended polarity of the energy delivered to the electrode.

23. A connector strap for providing electrical connection between an electronic stimulator and a pair of electrodes, said connector strap comprising:
first and second inner contacts;
first and second outer contacts;
a first conductor path between said first inner contact and said first outer contact, said first conductor path comprising a first diode;
a second conductor path between said first inner contact and said second outer contact, said second conductor path comprising a second diode;
a third conductor path between said second inner contact and said first outer contact, said third conductor path comprising a third diode; and
a fourth conductor path between said second inner contact and said second outer contact, said fourth conductor path comprising a fourth diode.

24. The connector strap of claim 23, wherein the first and second inner contacts comprise snap connectors for connection to the electronic stimulator.

25. The connector strap of claim 23, wherein the first and second outer contacts comprise snap connectors for connection to the electrodes.

26. A method of treating a body portion of a human or animal subject, comprising:
applying a garment electrode to a body portion to be treated; and
delivering therapeutic energy to the electrode from an electronic stimulator attached directly to the electrode.

27. The method of claim 26, further comprising applying compression to the body portion to be treated.

28. The method of claim 27, wherein gradient compression is applied to the body portion to be treated.
